(19) United States
(12) Patent Application Publication
(10) Pub. No.: US 2012/0328721 A1
(43) Pub. Date: Dec. 27, 2012

(54) ALOE VERA SPROUT CONCENTRATE OR EXTRACT HAVING SUPERIOR SKIN CELL GROWTH PROMOTION, ANTIOXIDANT, AND ANTI-ALLERGY EFFECTS

(75) Inventors: Tae Hyung Jo, Seongnam-si (KR); Seon Gil Do, Cheongju-si (KR); Eun Ju Shin, Yongin-si (KR); Hye In Jang, Suwon-si (KR); Jin Wan Kim, Songpa-gu (KR); Ghe Whan Ahn, Suwon-si (KR); Seung Won Yang, Cheongwon-gun (KR); Jin Hee Im, Cheongju-si (KR); Jeong Bum Nam, Cheongwon-gun (KR); Soo Gyeom Kim, Lyford, TX (US); Ken Jones, Edinburg, TX (US)

(73) Assignees: l; Hilltop Gardens Inc., Lyford, TX (US)

(21) Appl. No.: 13/509,704

(22) PCT Filed: Nov. 16, 2010
(86) PCT No.: PCT/KR10/08086

§ 371 Date: Aug. 22, 2012

(30) Foreign Application Priority Data
Nov. 16, 2009 (KR) 10-2009-0110554

(51) Int. Cl. A61K 36/886; A61P 17/00; A61Q 19/00; A61P 37/08; A61P 11/06; A61P 27/14; A61K 8/97; A61P 39/06

(52) U.S. Cl. 424/744

(57) ABSTRACT

The present invention relates to aloe vera sprout concentrate or extract having cell growth promotion, antioxidant, and anti-allergy effects, and to a skin cell growth promoting, antioxidant, and anti-allergy pharmaceutical composition and functional health food or cosmetics containing the aloe vera sprout concentrate or extract as an active ingredient.

### TECHNICAL FIELD

The present invention relates to an Aloe vera shoot concentrate or extract having the effects of promoting skin cell proliferation, antioxidant and anti-allergy, and a pharmaceutical composition for promoting skin cell proliferation, antioxidizing, or preventing or treating allergic disease or symptom, and a health functional food or cosmetic which comprises the same as an active ingredient.

### BACKGROUND ART

Aloe vera, a plant belonging to the family Liliaceae and genus Aloe, is classified as an evergreen perennial herb with fleshy leaves. The leaves are asymmetrical, in the shape of a semicircular column and spring from root and stem. The margin of the leaf has thorns in the shape of sharp sawteeth. The bottom of the leaf is broad, wraps around the stem and spreads in a rosette shape. The back of the leaf is round, and the front of the leaf is slightly dented.

From the past, Aloe vera has long been used as a folk medicinal herb. The first record for using aloe in a treatment is a clay tablet recorded by a Sumerian doctor in the year 2,100 BC. In addition, there are use records in the Chinese medical book “Gaeboboncho,” the Korean medical book “Donguibogam” and Korean pharmacopeia. Aloe has been used in the treatment of burns and dermal diseases (J. E. Crewe, M.D., Aloe in the Treatment of Burn and Scalds, Minnesota Medicine, 22, 538-539 [1985], Biltz J. J., Smith, J. W. and Gerard, J. R., Aloe vera gel in peptic ulcer therapy: Preliminary report. J. Am. Osteop. Assoc. 62, 731-735 [1963], Lee, L. M., Haggers, J. P., Robson, M. C. and Hahstrom, W. J., The therapeutic efficacy of Aloe vera cream in thermal injuries: Two case report., J. Am. Anim. Hosp. Assoc., 16, 768-777 [1980]), and its use area has recently been enlarged to cosmetics as well as health foods.

As the main ingredients of aloe, anthraquinones such as aloin, aloe-emodin, aloenin, aloesin and the like have been largely reported, and high-molecular polysaccharides, glycoproteins, amino acids and minerals are also known. As the medicinal effects of aloe ingredients, analgesic effect, anti-allergy, antibacterial activity, tonic effect, anti-inflammation, antitumor activity, blood glucose lowering and alleviation of cadmium toxicity have been reported. Specifically, skin cell regeneration, wound healing, protection of skin immunocyte from ultraviolet rays and skin moisturizing effect have been reported, so that aloe is used for a pharmaceutical composition for the improvement of skin wounds and skin protection, and cosmetics. However, such efficacies and ingredients are results derived from studies on the leaf of adult aloe which is grown for two (2) to three (3) years or more. There has been little study about baby aloe—i.e., Aloe vera shoot.

Aloe vera can be propagated by seed propagation, transplantation of shoot or planting the cutting of an adult aloe leaf Specifically, the most common propagation method is one in which shoots are flourishingly grown around an adult aloe for the purpose of propagation, and the shoots are then transplanted. However, only some of the shoots are transplanted to obtain grown aloes, and most shoots are removed to facilitate the growth of aloe. The removed shoots are discarded and not utilized for any commercial purpose. Little study has been performed about the efficacies and ingredients content of aloe shoot.

Plant shoots and baby plants have a number of blastocytes—which can be called stem cells of the plant, and it is known that cell proliferation is active. In addition, the production and secretion of plant growth hormone for facilitating plant growth (cell proliferation), and the generation of cellular building blocks for cell proliferation and differentiation are active, and there is a high content of structural proteins and functional proteins. Furthermore, tender young plants have a stronger self-defense system to protect themselves from the exterior environment than do adult plants, and are rich in antioxidant proteins.

Antioxidant action is a process to remove active oxygen species. Active oxygen species in the body are generated from general cell metabolism, and hydrogen peroxide (H₂O₂) is a representative substance. Such active oxygen species can impair cells and are regarded as a main cause of aging. In cells, catalase, superoxide dismutase (SOD) and the like convert hydrogen peroxide into oxygen and water to decrease impairment. However, because such conversion is not 100% complete, the remaining hydrogen peroxide adversely affects cells. To prevent it, antioxidants are widely used.

Allergy is a hypersensitive immune response to external foreign substances. Representative example is allergic rhinitis, allergic asthma, allergic dermatitis, allergic conjunctivitis or allergic gastrointestinal disease. In case of adult aloe, it has been reported that alprogen—which is one of the glycoproteins—is effective in the treatment of allergy by suppressing release of the inflammatory mediators, histamine and leukotriene, in a concentration-dependent manner.

### DETAILED DESCRIPTION OF THE INVENTION

### Technical Problem

The present invention relates to the utilization of Aloe vera shoot—which has been discarded as a byproduct in the cultivation of adult Aloe vera—by discovering that it has biological activities distinguishable from adult Aloe vera. The object of the present invention is to provide an Aloe vera shoot concentrate or extract which has the effects of promoting cell regeneration and cure healing, and has excellent antioxidant and anti-allergic effects, and a pharmaceutical composition for promoting skin cell proliferation, antioxidizing, or preventing or treating allergic disease or symptom, and a health functional food or cosmetic comprising the same.

### Solution to the Problem

To accomplish the above object, the present invention provides an Aloe vera shoot concentrate or extract having the effects of promoting skin cell proliferation, antioxidant and anti-allergy.

According to another aspect, the present invention provides an Aloe vera shoot concentrate having the effects of promoting skin cell proliferation, antioxidant and anti-allergy in which Aloe vera shoot—which is 12 months old or less—is crushed, anthraquinones and other pigments are removed, and then the crushed shoot is concentrated by conventional concentration methods.

According to still another aspect, the present invention provides an Aloe vera shoot concentrate having the effects of promoting skin cell proliferation, antioxidant and anti-allergy which is concentrated by methods such as vacuum evaporation, filtration concentration or freeze concentration.

According to still another aspect of the present invention, an Aloe vera shoot concentrate may be dried by spray drying, freeze drying, refractance window drying and other conventional drying methods.

According to still another aspect, the present invention provides an Aloe vera shoot extract having the effects of promoting skin cell proliferation, antioxidant and anti-allergy which is extracted with a solvent selected from the group consisting of water, C₁-C₄ anhydrous or hydrated lower alcohol, ketone having C₁-C₄ substituent, ester having C₁-C₄ substituent, chloroform and a mixture thereof

According to still another aspect, the present invention provides an Aloe vera shoot extract having the effects of promoting skin cell proliferation, antioxidant and anti-allergy which is extracted in a solvent of 1 to 20 times volume based on the weight of the Aloe vera shoot extract or a concentrate thereof.

According to still another aspect, the present invention provides an Aloe vera shoot extract having the effects of promoting skin cell proliferation, antioxidant and anti-allergy which is extracted by a method of hot water extraction, cold immersion extraction, ultrasonic extraction or reflux extraction or a concentrate thereof.

According to still another aspect, the present invention provides an Aloe vera shoot extract having the effects of promoting skin cell proliferation, antioxidant and anti-allergy which is extracted at 1 to 120° C. for 1 to 72 hours.

According to still another aspect, the present invention provides an extract in which an Aloe vera shoot is purified by using ultrafiltration or chromatography.

According to still another aspect, the present invention provides an Aloe vera shoot concentrate or extract which comprises 1-1,000 ppm or more of alprogen.

According to still another aspect, the present invention provides a promoting agent for skin cell proliferation comprising said Aloe vera shoot concentrate or extract as an active ingredient.

According to still another aspect, the present invention provides an antioxidant comprising said Aloe vera shoot concentrate or extract as an active ingredient.

According to still another aspect, the present invention provides a pharmaceutical composition for preventing or treating allergic disease or symptom comprising said Aloe vera shoot concentrate or extract as an active ingredient.

According to still another aspect, the present invention provides a pharmaceutical composition for preventing or treating allergic disease or symptom comprising said Aloe vera shoot concentrate or extract as an active ingredient in which said allergic disease or symptom is allergic rhinitis, allergic asthma, allergic dermatitis, allergic conjunctivitis or allergic gastrointestinal disease.

According to still another aspect, the present invention provides a health functional food for promoting skin cell proliferation comprising said Aloe vera shoot concentrate or extract as an active ingredient.

According to still another aspect, the present invention provides a health functional food for antioxidizing comprising said Aloe vera shoot concentrate or extract as an active ingredient.

According to still another aspect, the present invention provides a health functional food for alleviating or improving allergic disease or symptom comprising said Aloe vera shoot concentrate or extract as an active ingredient.

According to still another aspect, the present invention provides a skin cosmetic comprising said Aloe vera shoot concentrate or extract as an active ingredient.

### Effects of the Invention

An Aloe vera shoot concentrate or extract according to the present invention has high SOD activity and alprogen content, and shows excellent activities for promoting differentiation of keratinocyte, proliferation of fibroblast, collagen synthesis from fibroblast, and the inhibition of MMP-1 synthesis, compared with adult aloe. Therefore, when the Aloe vera shoot concentrate or extract according to the present invention is used as an active ingredient, a pharmaceutical composition, a health functional food or cosmetic having excellent effects of promoting skin cell proliferation, antioxidant and anti-allergy can be obtained.

### BRIEF DESCRIPTION OF THE DRAWING

FIG. 1 shows the alprogen content of Aloe vera shoot extract and adult Aloe vera extract according to the Example and the Comparative Example (Western blotting).

### MODES FOR CARRYING OUT THE INVENTION

The present inventors discovered that an Aloe vera shoot concentrate or extract according to the present invention has a high content of minerals, essential amino acids, crude proteins, glycoproteins and plant growth hormones. By conducting the subsequent studies, the present inventors found that the Aloe vera shoot concentrate or extract according to the present invention has high SOD activity and alprogen content, and shows excellent activities for promoting differentiation of keratinocyte, proliferation of fibroblast, collagen synthesis from fibroblast, and the inhibition of MMP-1 synthesis. As a result, the present inventors accomplished the invention directed to an Aloe vera shoot concentrate or extract having excellent effects for antioxidizing, reducing skin irritation, protecting skin tissue and improving skin texture, and a pharmaceutical composition, a health functional food and cosmetic comprising the same.

The present invention has its significance in that studies for aloe, which mainly have been carried out for adult aloe, are enlarged to Aloe vera shoot discarded in the cultivation of aloe, so that Aloe vera shoot can be commercially utilized, and it is discovered that Aloe vera shoot has excellent effects on skin regeneration, wound healing, antioxidizing action and allergy as compared with adult aloe.

It has been reported that the protein ingredient of aloe has effects on cell regeneration and wound healing. Measurement of the content of amino acids and crude proteins of the present Aloe vera shoot concentrate shows that is has a higher content than adult Aloe vera concentrate. In addition, it has been reported that plant growth hormones, which are known to be rich in shoots and juvenile plants, have effects on promoting fibroblast growth and wound healing (Abu Sinna G (1983) Comp. Biochem. Physiol. C. 74(2): 433-6., Robert H. Davis. Aloe vera: A scientific approach, Vantage). Because the Aloe vera shoot concentrate of the present invention has higher content of plant growth hormones than adult Aloe vera concentrate, it shows better effects of cell regeneration and wound healing as compared with adult Aloe vera concentrate.

Keratinocytes constitute 80% of epidermis, and repeat proliferation and differentiation with a cycle of a certain period. Shedding off in the stratum corneum is referred to as turn-over, and if it does not go smoothly, hyperkeratosis is caused or the stratum corneum is not properly formed. Fibroblasts synthesize extracellular matrix and collagen, and play an important role in wound healing. In addition, collagen is a constitutional element of skin and makes the skin firm and dense, along with elastin which is an elastic fiber. Matrix metalloproteinase (MMP)-1 is one of the major factors related to skin aging and wrinkle formation. MMP-1 causes wrinkle formation and a lowering of skin elasticity by destroying the connective tissue maintaining skin elasticity by degrading the ingredients that constitute extracellular matrix and basal membrane. In addition, MMP-1 promotes collagen degradation in the dermal layer and plays an important role in photoaging. Because the Aloe vera shoot concentrate of the present invention activates differentiation of keratinocytes and proliferation of fibroblasts, and has efficacy in inhibition of MMP-1 synthesis and promotion of collagen synthesis, it can promote skin cell proliferation, and its efficacy is excellent as compared with adult Aloe vera concentrate.

SOD protects the human body from active oxygen species by removing them from the human body. Specifically, in the skin SOD has effects on protecting and improving skin from harmful radiation and ultraviolet rays, and improving inflammatory reaction by microbes. It has been reported that Aloe vera has an excellent antioxidant effect by showing SOD activity on a level similar with spinach or liver (Sabeh E., 1996, Isozymes of Superoxide Dismutase from Aloe Vera. Enzyme Protein 49: 212-221). Because the Aloe vera shoot concentrate of the present invention has far higher content of SOD than adult Aloe vera concentrate, it shows excellent antioxidant activity.

In addition, it has been reported that alprogen of aloe has efficacy on treating allergy. Because the Aloe vera shoot concentrate of the present invention has higher content of alprogen than adult Aloe vera concentrate, it shows better anti-allergic efficacy.

The term “Aloe vera shoot” used herein refers to a baby Aloe vera which is 12 months old or less, preferably 6 months old or less and generated (germinated) from the base of adult Aloe vera.

The term “Aloe vera shoot concentrate” used herein refers to a concentrate prepared by crushing a harvested Aloe vera shoot, removing pigments and concentrating via conventional concentration methods such as vacuum evaporation, filtration concentration, spray drying, freeze drying, refractance window drying and the like, but includes those concentrated by methods within the scope of modification by a person skilled in the art.

The Aloe vera shoot concentrate of the present invention may be extracted by the following method. An Aloe vera shoot—which is 12 months old or less, preferably 6 months old or less—is harvested, washed with water and crushed. 0.1 to 10% of activated carbon based on total volume is added to the crushed Aloe vera shoot to adsorb anthraquinones and other pigments, and then the activated carbon is removed by filtering with a filter paper. The resultant is concentrated by conventional concentration methods and then dried by spray drying, freeze drying, refractance window drying or other general drying methods. However, concentration is not limited to the above methods, and other methods may be used.

The Aloe vera shoot concentrate of the present invention may be prepared by the following method. An Aloe vera shoot—which is 12 months old or less, preferably 6 months old or less—is cleanly washed and crushed. The crushed Aloe vera shoot is extracted with a solvent selected from the group consisting of water, alcohol, ketone, ester, halogenated hydrocarbon and a mixture thereof in about 1 to 20 times volume (ml) based on the weight (g) of the crushed Aloe vera shoot at room temperature to 100° C. for about 5 to 48 hours by a extraction method such as hot water extraction, cold immersion extraction, ultrasonic extraction, reflux extraction and the like repeated one time to five times. Then, the obtained extract is filtered, vacuum concentrated or freeze dried. The content of solvent in the final extract prepared as such is preferably less than 1%, more preferably less than 0.1%.

Preferably, the extraction solvent is selected from the group consisting of water, C₁-C₄ anhydrous or hydrated lower alcohol (e.g., methanol, ethanol, propanol, butanol and 1,3-butylene glycol, etc.), ketone having C₁-C₄ substituent (e.g., acetone, etc.), ester having C₁-C₄ substituent (e.g., ethyl acetate and butyl acetate, etc.), chloroform and a mixture thereof, more preferably C₁-C₄ hydrated lower alcohol, and most preferably ethanol. However, the extraction solvent is not limited thereto, and an extraction showing substantially the same effect may be obtained by using other extraction solvents.

Besides the extract using the above extraction solvents, the present invention includes an extract performed by a conventional purification process. The extract of the present invention may be used after purification by various additional methods such as isolation by an ultrafiltration membrane having a certain molecular weight cut-off value, or isolation by various chromatographies according to size, charge, hydrophobicity or affinity. The extract of the present invention may be prepared in a powder form by an additional process such as vacuum distillation, and freeze drying or spray drying. In the above chromatography, eluent may be preferably a mixed solvent in which a halogenated hydrocarbon solvent and lower alcohol are mixed in a ratio of 2:1 to 100:1, preferably 5:1 to 20:1, or a mixed solvent in which lower ester, lower alcohol and water are mixed in a ratio of 20:1:0 to 6:4:2.

One embodiment of the present invention provides an Aloe vera shoot concentrate or extract having the effects of promoting skin cell proliferation, antioxidant and anti-allergy.

Another embodiment of the present invention provides an Aloe vera shoot concentrate which is prepared by crushing Aloe vera shoot that is 12 months old or less, removing anthraquinones and other pigments, and then concentrating the resultant by a conventional concentration method.

Still another embodiment of the present invention provides an Aloe vera shoot concentrate in which a crushed Aloe vera shoot is concentrated by methods such as vacuum concentration, filtration concentration and freeze concentration.

In still another embodiment of the present invention, an Aloe vera shoot concentrate is dried by spray drying, freeze drying, refractance window drying and other conventional drying methods.

Still another embodiment of the present invention provides an Aloe vera shoot extract having the effects of promoting skin cell proliferation, antioxidant and anti-allergy which is extracted with a solvent selected from the group consisting of water, C₁-C₄ anhydrous or hydrated lower alcohol, ketone having C₁-C₄ substituent, ester having C₁-C₄ substituent, chloroform and a mixture thereof.

Preferably, the present invention provides an Aloe vera shoot extract having the effects of promoting skin cell proliferation, antioxidant and anti-allergy which is extracted with a solvent selected from the group consisting of water, C₁-C₄ anhydrous or hydrated lower alcohol and a mixture thereof, more preferably a solvent selected from the group consisting of C₁-C₄ hydrated lower alcohol and a mixture thereof, most preferably ethanol.

In still another embodiment of the present invention, said solvent is used in about 1 to 20 times volume (ml) based on the weight of crushed Aloe vera shoot (g).

Still another embodiment of the present invention provides an Aloe vera shoot extract having the effects of promoting skin cell proliferation, antioxidant and anti-allergy which is extracted by an extraction method of hot water extraction, cold immersion extraction, ultrasonic extraction or reflux extraction.

Still another embodiment of the present invention provides an Aloe vera shoot extract having the effects of promoting skin cell proliferation, antioxidant and anti-allergy in which an Aloe vera shoot is extracted by an extraction method of hot water extraction, cold immersion extraction, ultrasonic extraction or reflux extraction with said solvent at 1 to 120° C., preferably room temperature to 100° C.

Still another embodiment of the present invention provides an Aloe vera shoot extract having the effects of promoting skin cell proliferation, antioxidant and anti-allergy which is extracted by an extraction method of hot water extraction, cold immersion extraction, ultrasonic extraction or reflux extraction with said solvent for 1 to 72 hours, preferably 5 to 48 hours.

Still another embodiment of the present invention provides an Aloe vera shoot extract having the effects of promoting skin cell proliferation, antioxidant and anti-allergy in which the content of solvent in the extract obtained by said extraction method is less than 1%, preferably less than 0.1%.

Still another embodiment of the present invention provides an Aloe vera shoot extract having the effects of promoting skin cell proliferation, antioxidant and anti-allergy in which the content of alprogen in the extract obtained by said extraction method is 1 to 1,000 ppm or more.

In said chromatography, the eluent is preferably a mixed solvent in which a halogenated hydrocarbon solvent and lower alcohol are mixed in a ratio of 2:1 to 100:1, preferably 5:1 to 20:1, or a mixed solvent in which lower ester, lower alcohol and water are mixed in a ratio of 20:1:0 to 6:4:2.

Additionally, an extract of the present invention may be prepared in a powder form by a process such as vacuum distillation and freeze drying or spray drying.

The term “allergic disease or symptom” used herein includes, but is not limited to, anaphylactic shock, allergic rhinitis, allergic asthma, allergic dermatitis, allergic conjunctivitis or allergic gastrointestinal disease.

The term “prevention” means prophylactic administration of a preparation such as a pharmaceutical composition to healthy patients to prevent the outbreak of the diseases and symptoms mentioned herein. In addition, the term “prevention” means prophylactic administration of such a preparation to patients being in a pre-stage of the disease to be treated.

The term “treatment” used herein includes the prevention of a disease—i.e., the prophylactic administration of the combination, such as a combined preparation or pharmaceutical composition, to healthy patients to prevent the outbreak of the diseases and symptoms mentioned herein as well as the treatment of the diseases mentioned herein.

A pharmaceutical composition comprising the Aloe vera shoot concentrate or extract of the present invention as an active ingredient has the effects of promoting skin cell proliferation and antioxidant, and is useful in the treatment of allergy. For these purposes, the combinations of the present invention may be administered orally, parenterally (including subcutaneous injections, intravenous, intramuscular, intrastemal injection or infusion techniques) or rectally, in dosage unit formulations containing conventional non-toxic pharmaceutically acceptable carriers, adjuvants and vehicles.

The oral composition of the Aloe vera shoot concentrate or extract according to the present invention may additionally comprise inert constituents including pharmaceutically acceptable carriers, diluents, fillers, solubilizing or emulsifying agents and salts as is well known in the art and may be formulated into the forms of an oral dosage form such as powders, granules, tablets, capsules, suspensions, emulsions, syrups, aerosols; externals; suppositories and sterilized injectable solutions. For example, tablets used for combination therapy may be formulated in accordance with conventional procedures employing solid carriers that are well known in the art.

The term “pharmaceutically acceptable carrier” refers to ingredients other than active ingredients of composition, specifically a pharmaceutical composition. An example of pharmaceutically acceptable carrier may include lactose, dextrose, sucrose, sorbitol, mannitol, xylitol, erythritol, malditol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate and mineral oils. Additionally, the above pharmaceutically acceptable carrier may include, but is not limited to, diluents or additives such as fillers, extenders, binders, wetting agents, disintegrating agents, surfactants, etc.

The solid formulation for oral administration may be in the form of tablets, pills, powders, granules, capsules, etc. and may include at least one excipient such as starch, calcium carbonate, sucrose or lactose, and gelatin, and lubricants such as magnesium stearate, talc, etc. The liquid formulation for oral administration may be in the form of suspensions, solutions, emulsions, syrups, etc. and may include, but is not limited to, diluents such as water and liquid paraffin, wetting agents, sweeteners, aromatics or preservatives.

The formulation for parenteral administration may be in the form of sterilized solutions, non-aqueous solvents, suspensions, emulsions, lyophilizations or suppositories. Non-aqueous solvents and suspensions may include, but are not limited to, propylene glycol, polyethylene glycol, vegetable oils such as olive oil, injectable esters such as ethyl oleate, etc. Witepsol, macrogol, Tween 61, cacao oil, lauryl oil, glycerogelatin and the like as a carrier for suppositories may be used.

The food or health functional food of the present invention includes tablets, capsules, powders, granules, pills and the like for the purpose of protecting and improving the skin, protecting the human body and skin from active oxygen species, and improving allergic symptoms, but may be manufactured and processed in forms not limited thereto. The term “health functional food” used herein refers to food manufactured or processed with ingredients or components that possess the functionality useful for the human body according to the Korean Health Functional Food Act enacted by Act No. 6727, and the term “functionality” means gaining useful effect for health purposes by the adjustment of nutrients or the physiological effect, etc. for the structure and function of the human body.

The health functional food of the present invention may comprise conventional food additives. The suitability of the food additives is determined by the specification and standard of the concerned item in accordance with the General Provisions and General Test Methods of the Korea Food Additives Code authorized by the Korea Food and Drug Administration, unless otherwise specified.

The items listed in the above “Korea Food Additives Code” are, for example, synthetic additives such as ketones, glycine, potassium citrate, nicotinic acid, cinnamic acid and the like, natural additives such as persimmon color, licorice extract, microcrystalline cellulose, kaoliang color, guar gum and the like, and mixture additives such as ingredient containing L-sodium glutamate, alkali additives for noodles, preservatives, prepared tar dyes and the like.

For example, a health functional food in the form of a tablet may be prepared by granulating the mixture of Aloe vera shoot concentrate or extract, and excipients, binders, disintegrating agents and other additives with conventional methods, and compression molding with the addition of slip modifiers and the like, or directly compression molding the above mixture. In addition, the health functional food in the form of a tablet may comprise corrigents, or may be coated by coating agents, if necessary.

Among health functional foods in the form of a capsule, hard capsules may be prepared by charging the mixture of Aloe vera shoot concentrate or extract, and additives such as excipients and the like, or granules or coated granules thereof into a conventional hard capsule. Soft capsules may be prepared by charging the mixture of Aloe vera shoot extract and additives such as excipients and the like into a capsule base such as gelatin and the like. The soft capsules may comprise plasticizers such as glycerin or sorbitol, coloring agents or preservatives, if necessary.

A health functional food in the form of a pill may be prepared by moulding the mixture of Aloe vera shoot concentrate or extract, and excipients, binders, disintegrating agents and the like with a proper method. The pills may be coated with white sugar or other proper coating agents, or sparkling with a powder of starch, talc or proper substances, if necessary.

A health functional food in the form of granules may be prepared in a granular shape by treating the mixture of Aloe vera shoot concentrate or extract, and excipients, binders, disintegrating agents and the like with a proper method. The granules may comprise fragrance ingredients, corrigents and the like, if necessary.

The definitions of the excipients, binders, disintegrating agents, slip modifiers, corrigents, fragrance ingredients and the like are described in publications known in the art and include those having the same or similar function (Korean Pharmacopeia explanation, MoonSung Publications, Korean Council of Pharmaceutical Colleges, fifth edition, pp. 33-48, 1989).

The cosmetic composition for protecting and improving the skin according to the present invention may be used in external medicines, cosmetics and the like, and formulated into any formulations conventionally prepared in the art. The “cosmetics” of the present invention include, but are not limited to, ointments, tinctures, soaps and general cosmetics—for example, cream, massage cream, cold cream, cleansing cream, skin lotion, milk lotion, nutrition cream, moisture cream, essence, pack, foundation, white powder, lipstick, sunblock cream, bathing agent, facial cleansing cream, facial cleansing gel, facial cleansing foam, body shampoo, body gel, body cream, hand cream and the like, and may be variously formulated.

For formulation, they are prepared by conventional methods used in the manufacture of cosmetics by using adjuvants and excipients—which are conventionally used in external medicines, soap basis and cosmetics manufacturing field—for example, emulsifiers, preservatives, lubricants, diluents, thickeners, humectants, pigments, antiseptics, fragrances and the like. As a humectant, one or more moisturizing components—which include, but are not limited to, ethylene glycol, diethylene glycol, triethylene glycol, polyethylene glycol, propylene glycol, dipropylene glycol, 1,3-butylene glycol, glycerin, sorbitol and collagen—may be used alone or in combination. The example of emulsifier includes, but is not limited to, cyclomethicone, polymethyl methacrylate or hydrogenated castor oil. The example of thickener includes, but is not limited to, methyl cellulose, carboxymethyl cellulose, carboxymethyl hydroxyguanine, hydroxymethyl cellulose, hydroxyethyl cellulose, carboxyvinyl polymer, polyquaternium, cetearyl alcohol, stearic acid and carrageenan. The example of antiseptic includes, but is not limited to, imidazolidinyl urea, methylparaben and propylparaben.

The preparation processes of the Aloe vera shoot concentrate of the present invention and adult Aloe vera concentrate are explained in the Example and the Comparative Example. In addition, the present invention is explained in greater detail via the following Experimental Examples in which the contents of crude proteins, amino acids, minerals, plant growth hormones and alprogen, SOD activity, activity for the promotion of keratinocyte differentiation, activity for the promotion of fibroblast proliferation, inhibitory activity against MMP-1 synthesis and activity for the promotion of collagen synthesis of the present Aloe vera shoot concentrate and adult Aloe vera concentrate are compared. However, the Example and Experimental Examples merely set forth to explain the present invention in more detail, and it must be understood that the protection scope of the present invention is not limited thereto.

### EXAMPLE 1

### Preparation of Aloe Vera Shoot Concentrate

Aloe vera shoot, 6 months old or less, cultivated in Hainan, China was harvested, washed, and then anthraquinones and other pigments were removed by using an activated carbon column. The resultant was filtered, vacuum concentrated and freeze dried to give a powdery sample. The obtained sample was stored at room temperature.

### COMPARATIVE EXAMPLE 1

### Preparation of Adult Aloe Vera Concentrate

The leaf of Aloe vera, 2 to 3 years old, cultivated in Hainan, China was harvested and processed according to the same methods as Example 1.

### EXPERIMENTAL EXAMPLE 1

### Comparison of Crude Proteins Content between Aloe Vera Shoot Concentrate and Adult Aloe Vera Concentrate

The content of crude proteins was measured according to the semimicro Kjeldahl method of the Korean Health Functional Food Code (2008-64) III.3.3.1.

As a result, as can be seen from Table 1, the content of crude proteins of Aloe vera shoot concentrate is about twice as high as of adult Aloe vera concentrate.

TABLE 1

| | Example 1 | Comparative Example 1 |
|---|---|---|
| Crude proteins content (%) | 3.918 | 2.032 |

### EXPERIMENTAL EXAMPLE 2

### Comparison of Amino Acids Content Between Aloe Vera Shoot Concentrate and Adult Aloe Vera Concentrate

The content of amino acids was measured according to the amino acid analysis method of the Korean Health Functional Food Code (2008-64) III.3.3.2.

As a result, as can be seen from Table 2, the content of all amino acids except for leucine, which is slightly lower, of Aloe vera shoot concentrate is higher than that of adult Aloe vera concentrate. In addition, the total content of amino acids of Aloe vera shoot concentrate is 3 times or more higher than that of adult Aloe vera concentrate.

TABLE 2

| | Example 1 (%) | Comparative Example 1 (%) |
|---|---|---|
| leucine | 0.073 | 0.034 |
| lysine | 0.107 | 0.031 |
| methionine | 0.006 | 0.005 |
| valine | 0.100 | 0.045 |
| isoleucine | 0.062 | 0.069 |
| threonine | 0.073 | 0.043 |
| tryptophane | 0.025 | 0.026 |
| histidine | 0.026 | 0.026 |
| phenylalanine | 0.030 | 0.030 |
| glysine | 0.093 | 0.045 |
| alanine | 0.186 | 0.142 |
| asparagine | 0.087 | 0.088 |
| aspartic acid | 0.390 | 0.040 |
| glutamine | 0.564 | 0.565 |
| serine | 0.115 | 0.063 |
| tyrosine | 0.045 | 0.042 |
| cysteine | 0.002 | 0.001 |
| Total | 1.852 | 1.295 |

### EXPERIMENTAL EXAMPLE 3

### Comparison of Minerals Content

The test was carried out according to the minerals analysis method of the Korean Health Functional Food Code (2008-64) III.3.2. As a result, as can be seen from Table 3, the Aloe vera shoot concentrate contains 30% or more higher minerals than the adult Aloe vera concentrate.

TABLE 3

| | Example 1 (%) | Comparative Example 1 (%) |
|---|---|---|
| Ba | 0.01 | 0.01 |
| B | 0.01 | 0.01 |
| Ca | 2.84 | 3.47 |
| K | 9.14 | 5.11 |
| Mg | 1.24 | 1.02 |
| Mn | 0.06 | 0.04 |
| Na | 0.53 | 0.97 |
| P | 0.40 | 0.30 |
| Si | 0.02 | 0.03 |
| Zn | 0.01 | 0.02 |
| Total | 14.26 | 10.98 |

### EXPERIMENTAL EXAMPLE 4

### Comparison of Plant Growth Hormone Content

The content of plant growth hormone, indoleacetic acid (IAA), was quantitatively measured via competitive ELISA by using Phytodetek IAA test kit.

As a result, as can be seen from Table 4, the content of IAA of Aloe vera shoot concentrate is about 60% higher than that of adult Aloe vera concentrate.

TABLE 4

| | Example 1 | Comparative Example 1 |
|---|---|---|
| IAA | 2125.4 | 1332.7 |

### EXPERIMENTAL EXAMPLE 5

### Comparison of Alprogen Content

20 g of Example 1 and Comparative Example were dissolved in 175 ml of extraction buffer, respectively. The obtained solutions were salted out with ammonium sulfate and desalted to give samples. The obtained samples were concentrated. 0.5 ng of samples were electrophoresed by SDS-PAGE, and then western blotting was carried out by using anti-alprogen antibodies to measure the content of alprogen.

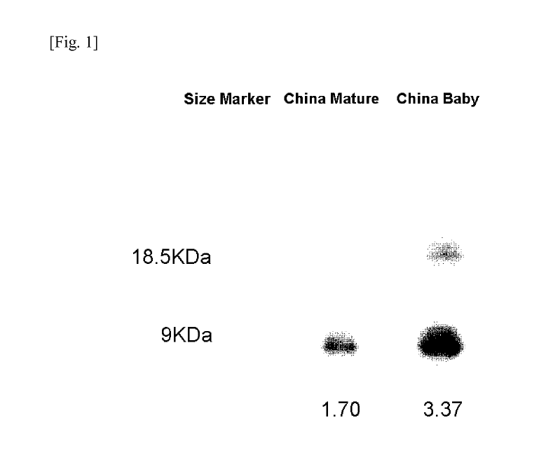

As a result, as can be seen from FIG. 1 and Table 5, the content of alprogen of Aloe vera shoot concentrate is higher than that of adult Aloe vera concentrate.

TABLE 5

| | Example 1 | Comparative Example 1 |
|---|---|---|
| Alprogen | 1.678 | 1.337 |

### EXPERIMENTAL EXAMPLE 6

### Comparison of SOD Activity

SOD activity was measured according to the SOD test method on pages 101-103 of the Korean Health Functional Food Code (2004).

As a result, as can be seen from Table 6, the SOD activity of Aloe vera shoot concentrate is 9600.1 units per gram (g), whereas that of adult Aloe vera concentrate is 1361.71 units per gram (g). Therefore, the SOD activity of Aloe vera shoot concentrate is 7 times or more higher than that of adult Aloe vera concentrate.

TABLE 6

| | Example 1 | Comparative Example 1 |
|---|---|---|
| SOD (unit/g) | 9600.1 | 1361.71 |

### EXPERIMENTAL EXAMPLE 7

### Comparison of Activity for the Promotion of Keratinocyte Differentiation

To evaluate the effect on the promotion of keratinocyte differentiation, the amount of CE (cornified envelope) which is generated at the time of differentiation of keratinocytes was measured by absorbance (refer to Korean Patent Application No. 10-2005-0076551).

After human keratinocyte primary culture was adhered to culture flasks, the cells were treated with Example 1 and Comparative Example 1 at the concentration of 1 ppm, respectively, and then cultured to be 70-80% confluent for 5 days. The low-calcium (0.03 mM) treating group and the high-calcium (1.2 mM) treating group were used as a negative control and a positive control, respectively.

The cultured cells were harvested, washed with PBS (phosphate buffered saline), and 1 ml of 10 mM Tris-HCl (pH 7.4) containing 2% SDS (sodium dodecyl sulfate) and 20 mM DTT (dithiothreitol) was added thereto. The obtained solution was sonicated, and then the protein content was measured to evaluate the extent of cell differentiation.

As a result, as can be seen from Table 7, the Aloe vera shoot concentrate shows 40% higher activity for keratinocyte differentiation as compared with the negative control, and about 27% higher activity than the adult Aloe vera concentrate.

TABLE 7

| | Activity for keratinocyte |
|---|---|
| | differentiation (%) |
| Low-calcium (0.03 mM) solution | 100 |
| (Negative control) | |
| Example 1 | 140 |
| Comparative Example 1 | 110 |
| High-calcium (1.2 mM) solution | 213 |
| (Positive control) | |

### EXPERIMENTAL EXAMPLE 8

### Comparison of Activity for Promotion of Fibroblast Proliferation Between Aloe Vera Shoot Concentrate and Adult Aloe Vera Concentrate

To evaluate the activity for the promotion of cell growth, human skin fibroblast was cultured, and then cell viability was measured by the MTT method (3-(4,5-dimethylthiazol-2-yl)-2,5-diphenyltetrazolium bromide reduction test) according to the publication (A. A. Van de Loosdrecht, E. Nennie, G. J. Ossenkoppele, and R. H. Beelen, Langenhuijsen M M. J. Immunol. Methods, 141(1), p. 15, 1991) to discern whether each sample has the activity for the promotion of fibroblast proliferation.

As a result, as can be seen from Table 8, the Aloe vera shoot concentrate shows higher cell viability than the adult Aloe vera concentrate at all tested concentrations. Specifically, at the concentration of 10 ng/ml the Aloe vera shoot concentrate promotes 44.32% of cell proliferation as compared with the negative control in which cells were not treated with the sample.

TABLE 8

| Concentrate | Cell viability (%) |
|---|---|
| Example 1 (1 μg/ml) | 105.12 |
| Example 1 (5 μg/ml) | 121.23 |
| Example 1 (10 μg/ml) | 144.32 |
| Comparative Example 1 (1 μg/ml) | 101.25 |
| Comparative Example 1 (5 μg/ml) | 105.74 |
| Comparative Example 1 (10 μg/ml) | 113.94 |

### EXPERIMENTAL EXAMPLE 9

### Comparison of Inhibitory Activity Against MMP-1 Synthesis

To evaluate the inhibitory activity against MMP-1 gene expression of Aloe vera shoot concentrate, the change of mRNA expression was measured. RNA was extracted by using Trizol (Invitrogen, USA) according to the guanidium thiocyanate-phenol-chloroform extraction method, and then RT-PCR was carried out by using an All-in-one RT-PCR kit (SuperBio, Korea). PCR (reverse transcription at 50° C. for 30 minutes, inactivation of reverse transcriptase at 96° C. for 3 minutes, 25 cycles of 94° C. for 1 minute, 48° C. for 1 minute and 72° C. for 1 minute) was carried out by using an MMP-1 primer of Table 9, and actin was used as a control group.

TABLE 9
MMP-1 primer

| | |
|---|---|
| Sense | 5′-TGGGAGCAAACACATCTGA-3′ |
| Antisense | 5′-ATCACTTCTCCCCGAATCGT-3′ |

As a result, as can be seen from Table 10, the Aloe vera shoot concentrate inhibits 53.5% of MMP-1 expression as compared with the control group, and shows about 30% of additional inhibitory activity even compared with the adult Aloe vera concentrate.

TABLE 10
Effect on inhibition of mRNA expression

| | MMP-1 expression (%, corrected |
|---|---|
| | value based on actin) |
| Control group | 100 |
| Comparative Example 1 | 75.5 |
| Example 1 | 46.5 |

### EXPERIMENTAL EXAMPLE 10

### Comparison of Activity for Promotion of Collagen Synthesis

To evaluate the activity for the promotion of collagen synthesis of Aloe vera shoot concentrate, the expression of collagen mRNA was measured. RT-PCR was carried out with the same method as Experimental Example 9 except that the condition was 30 cycles of 94° C. for 1 minute, 62° C. for 1 minute and 72 ° C. for 1 minute, and collagen primer of Table 11 was used.

TABLE 11
Collagen primer

| | |
|---|---|
| Sense | 5′-CTGGCAAAGAAGGCGGCAAA-3′ |
| Antisense | 5′-CTCACCACGATCACCACTCT-3′ |

As a result, as can be seen from Table 12, treatments of 100 μg/ml and 200 μg/ml of Aloe vera shoot concentrate increase the expression of type I collagen by 31.56% and 53.96%, respectively. Such results are about 23% and 26% increased values as compared with the adult Aloe vera concentrate, and the positive control shows the similar increased rate.

TABLE 12
Expression amount of type I collagen mRNA

| | Type I collagen expression amount (%) | |
|---|---|---|
| | 100 μg/ml | 200 μg/ml |
| Example 1 | 108.59 | 127.59 |
| Comparative Example 1 | 131.56 | 153.96 |
| Control | 131.1 | 156.3 |
| (vitamin C 100 μg/ml) | | |
| Non-treatment group | 100 | 100 |

The composition of the present invention may be formulated as the following formulations. However, the following formulations merely exemplify the present invention, and the protection scope of the present invention is not limited thereto.

### FORMULATION EXAMPLE 1

### Skin Lotion

After 1% by weight of Tween 20 was dissolved by heating to about 60° C., a fragrance ingredient was added thereto and agitated. 3% by weight of glycerin, 2% by weight of butylene glycol, 2% by weight of propylene glycol and an antiseptic were added thereto and agitated to dissolve, and 80% by weight of purified water was added thereto. Finally, 2% by weight of Aloe vera shoot concentrate and 10% by weight of ethanol were added thereto and sufficiently agitated to give the title product.

### FORMULATION EXAMPLE 2

### Milk Lotion

1.5% by weight of sitosterol, 1.5% by weight of polyglyceryl-2 oleate, 1% by weight of ceramide, 1% by weight of ceteareth-4 and 1.5% by weight of cholesterol were homogenized at a certain temperature. 2% by weight of Aloe vera shoot concentrate, 0.4% by weight of dicetyl phosphate, 4.0% by weight of concentrated glycerin, 10% by weight of sunflower oil, 0.3% by weight of carboxyvinyl polymer, 0.3% by weight of xanthan gum and an antiseptic were heated and dissolved with a conventional method. 76.5% by weight of purified water was added thereto. The obtained solution was emulsified by a homogenizer, and then a suitable amount of fragrance agent was added and re-agitated to give the title product.

### FORMULATION EXAMPLE 3

### Nutrition Essence

1.7% by weight of sitosterol, 1.5% by weight of polyglyceryl-2 oleate, 0.7% by weight of ceramide, 1.2% by weight of ceteareth-4 and 1.5% by weight of cholesterol were homogenized at a certain temperature. 5% by weight of Aloe vera shoot concentrate, 0.4% by weight of dicetyl phosphate, 5.0% by weight of concentrated glycerin, 15% by weight of sunflower oil, 0.2% by weight of carboxyvinyl polymer, 0.2% by weight of xanthan gum and an antiseptic were heated and dissolved with a conventional method. 67.6% by weight of purified water was added thereto. The obtained solution was emulsified by a homogenizer, and then a suitable amount of fragrance agent was added and re-agitated to give the title product.

### FORMULATION EXAMPLE 4

### Health Functional Food, Liquid

50% by weight of purified water was incorporated into an agitation tank, and heated and agitated. 0.34% by weight of Caragel was added thereto and heated to 95±3° C. of content temperature to completely dissolve. After dissolution, the primary ingredients (1-5% by weight of Aloe vera shoot concentrate, 6% by weight of fructooligosaccharide and 0.1% by weight of potassium sorbate) were sequentially added while maintaining 80±2° C. of content temperature and agitated to homogeneously dissolve.

After dissolution, the temperature of content was cooled to 75±5° C., and then the secondary ingredients (0.03% by weight of green tea extract powder, 0.1% by weight of licorice powder, 0.5% by weight of Japanese apricot concentrate and 0.05% by weight of citric acid [anhydrous]) were added thereto and agitated at 75±5° C. for 30 minutes to give the title product.

### FORMULATION EXAMPLE 5

### Health Functional Food, Granule

The weighed ingredients (28% by weight of Aloe vera shoot concentrate, 25% by weight of xylitol, 23% by weight of fructooligosaccharide powder, 0.1% by weight of xanthan gum, 0.1% by weight of alpha corn, 0.5% by weight of inositol, 0.05% by weight of fermented soybean extract powder, 0.1% by weight of rice germ extract, 0.2% by weight of corn starch, 0.5% by weight of vitamin C and 0.1% by weight of sucrose esters of fatty acids) were incorporated into a fluid bed granulator, mixed for 10 minutes, and then granulated by the automatic system of the fluid bed granulator to give the title product.

### FORMULATION EXAMPLE 6

### Health Functional Food, Tablet

Ingredients (30.0% by weight of Aloe vera shoot concentrate, 10.0% by weight of fine flour, 30.0% by weight of dextrin, 20.0% by weight of fructooligosaccharide, 8.94% by weight of lactobacillus powder, 1.0% by weight of vitamin C and 0.05% by weight of apple flavor powder) were incorporated into a mixer and primary mixing was carried out. After the addition of flow agent (0.01% by weight of sucrose esters of fatty acids), secondary mixing was carried out and then the obtained mixture was tableted by a tablet machine to give the title product.

### FORMULATION EXAMPLE 7

### Health Functional Food, Capsule

60.0% by weight of soybean oil, 2.0% by weight of lecithin and 1.0% by weight of beeswax were added to a mixing tank, and then mixed with emulsifying by heating to 70±5° C. . The resultant was cooled to 40±5° C., and then the weighed ingredients (30.0% by weight of Aloe vera shoot concentrate, 7% by weight of chondroitin sulfate, 2.0% by weight of green lipped mussel powder) were sequentially added thereto and homogenously mixed by using a mixer for 1 hour. The obtained mixture was agitated with heating to 70-80° C. to dissolve. After dissolution, the resultant was degassed in conventional conditions to give the title product.

1. An Aloe vera shoot concentrate or extract.

2. The Aloe vera shoot concentrate according to claim 1, which is concentrated by a vacuum evaporation, a filtration concentration or a freeze concentration.

3. The Aloe vera shoot extract according to claim 1, which is extracted with a solvent selected from the group consisting of water, C₁-C₄ anhydrous or hydrated lower alcohol, ketone having C₁-C₄ substituent, ester having C₁-C₄ substituent, chloroform and a mixture thereof.

4. The Aloe vera shoot concentrate or extract according to claim 1, which comprises 1-1,000 ppm or more of alprogen.

5. The concentrate or extract according to claim 1, which is in a dried form.

6. A pharmaceutical composition for promoting skin cell proliferation, antioxidizing, or preventing or treating allergic disease or symptom comprising an Aloe vera shoot concentrate or extract as defined in claim 1, as an active ingredient.

7. The pharmaceutical composition according to claim 6, wherein the allergic disease or symptom is anaphylactic shock, allergic rhinitis, allergic asthma, allergic dermatitis, allergic conjunctivitis or allergic gastrointestinal disease.

8. A health functional food comprising an Aloe vera shoot concentrate or extract as defined in claim 1, as an active ingredient.

9. A skin cosmetic comprising an Aloe vera shoot concentrate or extract as defined in claim 1 as an active ingredient.

10. A method of promoting skin cell proliferation, imparting antioxidant effect, or treating allergies comprising administering the Aloe vera shoot concentrate or extract as defined in claim 1.


SEQUENCE LISTING (4 sequences)

SEQ ID NO: 1 - DNA, 19 bp, artificial
  Sense primer for RT-PCR of MMP-1
tgggagcaaacacatctga

SEQ ID NO: 2 - DNA, 20 bp, artificial
  Antisense primer for RT-PCR of MMP-1
atcacttctccccgaatcgt

SEQ ID NO: 3 - DNA, 20 bp, artificial
  Sense primer for RT-PCR of collagen
ctggcaaagaaggcggcaaa

SEQ ID NO: 4 - DNA, 20 bp, artificial
  Antisense primer for RT-PCR of collagen
ctcaccacgatcaccactct